(19) United States
(12) Patent Application Publication
(10) Pub. No.: US 2015/0023475 A1
(43) Pub. Date: Jan. 22, 2015

(54) APPARATUS AND METHOD FOR MOBILE X-RAY IMAGING

(71) Applicant: C-ARM BUDDIES, Tyler, TX (US)

(72) Inventors: Charles R. Gordon, Tyler, TX (US); James E. Deaton, Georgetown, TX (US); Blake Brookshire, Nashville, TN (US)

(21) Appl. No.: 14/334,182

(22) Filed: Jul. 17, 2014

Related U.S. Application Data
(60) Provisional application No. 61/847,261, filed on Jul. 17, 2013.

(51) Int. Cl. A61B 6/00

(52) U.S. Cl. CPC A61B 6/4405; A61B 6/461; A61B 6/548
USPC 378/195

(57) ABSTRACT

Apparatus and methods for obtaining repeatable images of a patient are disclosed herein. Mechanical fixtures may be attached to a mobile imaging device and a patient support platform to allow them to be placed at a fixed relationship to one another. Additionally, devices for measuring movement of the mobile imaging device with respect to the patient support platform are disclosed.

### CROSS-REFERENCE TO RELATED APPLICATIONS

This application claims priority to U.S. Provisional Application No. 61/847,261 filed Jul. 17, 2013, the entire contents of which is specifically incorporated by reference herein without disclaimer.

### BACKGROUND

1. Field of the Invention

The present invention relates generally to imaging systems. More particularly, the present invention relates to mobile C-arm imaging systems.

2. Description of Related Art

Certain medical procedures are typically conducted using C-arm imaging systems. For example, spinal fusion surgery typically involves taking multiple images using a C-arm x-ray machine. C-arm imaging systems may be permanently affixed to the structure of the hospital or may be mounted on a mobile base. Typically, mobile systems are manually maneuvered into a surgery or operating room.

Once a C-arm imaging system is placed into position adjacent a patient, images are taken of the patient's anatomy at a desired location. The acquisition of a suitable image involves skill and expertise by an operator, who is typically a radiology technician. The operator must properly place the C-arm, which involves moving the base into the proper position, configuring the C-arm to place the X-ray emitter and receptor in desired locations, and adjusting the imaging properties (dosage, collimation, etc.) to obtain an image. Typically, multiple images are taken and the C-arm is manually maneuvered into different positions to produce images at different views and positions. After enough “before” images are obtained, medical procedures (e.g., surgery) are performed and “after” images are obtained to ensure that the procedures were successful. It is desirable that the before and after images are taken from the same position so that they can be easily compared. Reproducing the exact same positioning is difficult, however, with existing systems, and involves a significant amount of a radiologic technician's time.

### SUMMARY

An system and method for obtaining reproducible images of a patient are disclosed herein.

In one exemplary embodiment, a device for taking images of a patient on a support platform with a mobile imaging device, comprises a platen configured to be coupled to the support platform in a fixed position and a locating fixture configured to be coupled to the mobile imaging device in a fixed position. The platen and locating fixture are adapted to be selectively coupled to one another in a fixed relationship.

The platen may be slidable along the longitudinal axis of the support platform.

The support platform may comprise a longitudinal slot, and the platen comprises a key for interfacing with the longitudinal slot.

The longitudinal slot may comprise a dovetail slot and the key may comprise a dovetail key.

The platen may comprise a mating surface with apertures and the locating fixture may comprise pins for engaging the apertures.

The device may comprise a locking apparatus for locking the locating fixture into position with respect to the platen.

The device may comprise a second platen coupled to the support platform for selective coupling with the locating fixture.

In another exemplary embodiment, an imaging device comprises a mobile base for supporting an imaging apparatus and instrumentation for measuring displacement of the mobile base with respect to a patient support platform.

The instrumentation may comprise one or more of encoders coupled to wheels coupled to the mobile base, a laser displacement instrument, an inertial measurement sensor, a gyroscope and an accelerometer, or a video positioning system.

The device may comprise a remote operation controller for operating the imaging apparatus by a remote user.

The device may comprise a memory for storing at least one location of the mobile base, a powered transport for moving the mobile base, and a controller for controlling the powered transport to move the mobile base to the at least one stored location. The controller may be operated by a remote user.

The imaging device may comprise a C-arm, and the memory may be configured to store articulation information regarding the position of the C-arm.

The memory may be configured to store imaging properties of the imaging apparatus. The imaging properties may comprise an amount of x-ray energy delivered and a collimation of an image.

The controller may be configured to move the mobile base to a storage position.

The at least one location may comprise a plurality of locations, and the controller may move the mobile base to a selected one of the plurality of locations.

A memory for storing at least one location of the mobile base and a display for indicating the position of the mobile base with respect to the at least one location may be provided. The at least one location may comprise a plurality of locations, and the display may indicate the position of the mobile base with respect to a selected one of the plurality of locations.

A memory for storing at least one location of the mobile base, and a steering mechanism for guiding the mobile base toward the at least one stored location may be provided.

The at least one location may comprise a plurality of locations, and the steering mechanism guides the mobile base toward a selected one of the plurality of locations.

In accordance with another illustrative embodiment, a method of taking surgical images comprises positioning a mobile imaging device at a first location for taking images, detecting information regarding the movement of the mobile imaging device from the first location to a second location, and providing visual guidance on a display device representing the distance between the current location of the mobile imaging device and the recorded first location of the mobile imaging device.

The method may further comprise storing imaging information associated with the obtained images, recalling the stored imaging information, and obtaining additional images using the stored imaging information.

The step of detecting information may comprise sensing movement using an inertial measurement sensor.

The step of detecting information may comprise obtaining visual images of surrounding objects.

The step of detecting information may comprise detecting movement of wheels associated with the mobile imaging device.

The step of detecting information may comprise scanning surrounding objects with a laser.

In accordance with yet another illustrative embodiment, a method of taking surgical images comprises positioning a mobile imaging device at a first location for taking images, detecting information regarding the movement of the mobile imaging device from the first location to a second location, and returning the mobile imaging device to the first location utilizing the detected information.

The method may further comprise storing imaging information associated with the obtained images, recalling the stored imaging information, and obtaining additional images using the stored imaging information.

The step of detecting information may comprise sensing movement using an inertial measurement sensor.

The step of detecting information may comprise obtaining visual images of surrounding objects.

The step of detecting information may comprise detecting movement of wheels associated with the mobile imaging device.

The step of detecting information may comprise scanning surrounding objects with a laser.

The term “coupled” is defined as connected, although not necessarily directly. The terms “a” and “an” are defined as one or more unless this disclosure explicitly requires otherwise.

The terms “substantially,” “approximately,” and “about” are defined as largely but not necessarily wholly what is specified (and includes what is specified; e.g., substantially 90 degrees includes 90 degrees and substantially parallel includes parallel), as understood by a person of ordinary skill in the art. In any disclosed embodiment, the terms “substantially,” “approximately,” and “about” may be substituted with “within [a percentage] of” what is specified, where the percentage includes 0.1, 1, 5, and 10 percent.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a system, or a component of a system, that “comprises,” “has,” “includes” or “contains” one or more elements or features possesses those one or more elements or features, but is not limited to possessing only those elements or features. Likewise, a method that “comprises,” “has,” “includes” or “contains” one or more steps possesses those one or more steps, but is not limited to possessing only those one or more steps. Additionally, terms such as “first” and “second” are used only to differentiate structures or features, and not to limit the different structures or features to a particular order.

A device, system, or component of either that is configured in a certain way is configured in at least that way, but it can also be configured in other ways than those specifically described.

Any embodiment of any of the systems and methods can consist of or consist essentially of—rather than comprise/include/contain/have—any of the described elements, features, and/or steps. Thus, in any of the claims, the term “consisting of” or “consisting essentially of” can be substituted for any of the open-ended linking verbs recited above, in order to change the scope of a given claim from what it would otherwise be using the open-ended linking verb.

The feature or features of one embodiment may be applied to other embodiments, even though not described or illustrated, unless expressly prohibited by this disclosure or the nature of the embodiments.

Details associated with the embodiments described above and others are presented below.

### BRIEF DESCRIPTION OF THE DRAWINGS

FIG. 1 is an end view of a mobile imaging device with a mechanical fixture for obtaining repeatable images in accordance with an exemplary embodiment;

FIG. 2 is a side view of a portion of a surgical bed for use with the mobile imaging device of FIG. 1 in accordance with an exemplary embodiment;

FIG. 3 is a top view of a mobile imaging device utilizing rotational position and angular position encoders in accordance with an exemplary embodiment;

FIG. 4 is a top view of a mobile imaging device with a powered drive mechanism in accordance with an exemplary embodiment;

FIG. 5 is a top view of a mobile imaging device utilizing a laser displacement instrument in accordance with an exemplary embodiment;

FIG. 6 is a top view of a mobile imaging device utilizing an inertial measurement sensor in accordance with an exemplary embodiment; and

FIG. 7 is a top view of a mobile imaging device utilizing a video positioning sensor in accordance with an exemplary embodiment.

### DESCRIPTION OF ILLUSTRATIVE EMBODIMENTS

In the following detailed description, reference is made to the accompanying drawings, in which are shown exemplary but non-limiting and non-exhaustive embodiments of the invention. These embodiments are described in sufficient detail to enable those having skill in the art to practice the invention, and it is understood that other embodiments may be used, and other changes may be made, without departing from the spirit or scope of the invention. The following detailed description is, therefore, not to be taken in a limiting sense, and the scope of the invention is defined only by the appended claims. In the accompanying drawings, like reference numerals refer to like parts throughout the various figures unless otherwise specified.

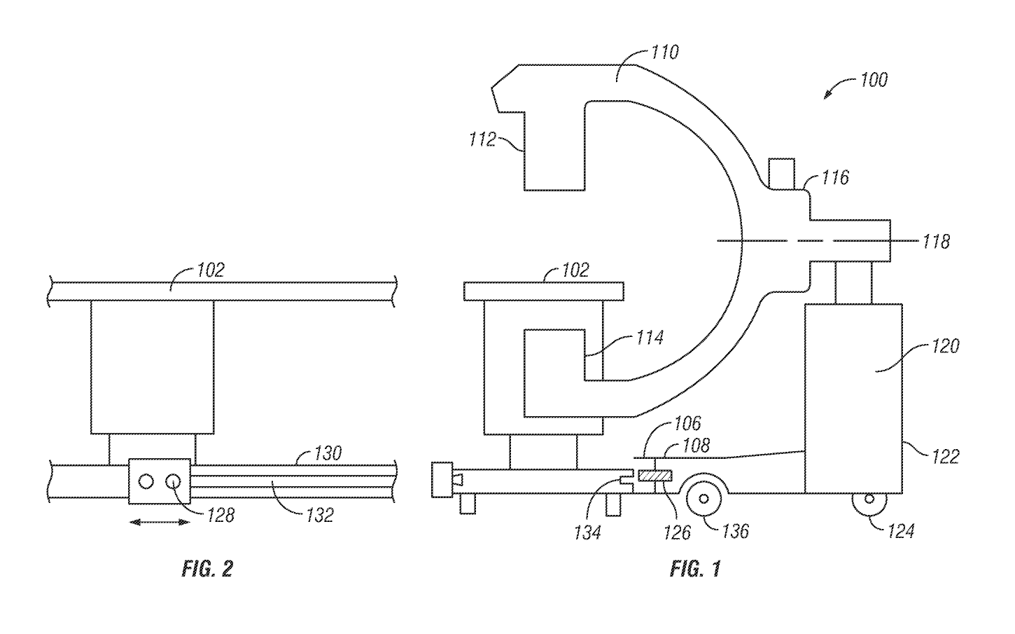

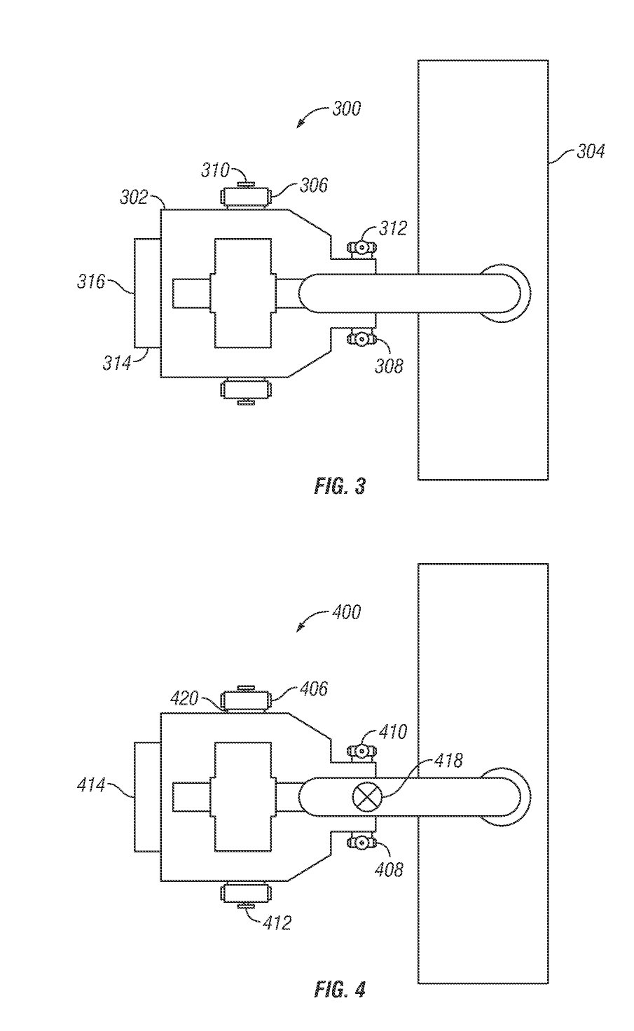

Referring to FIG. 1, in accordance with an illustrative embodiment, a mobile imaging device 100 for taking images of a patient on a support platform 102 comprises a platen 106 and a locating fixture 108. The platen 106 is configured to be selectively coupled to the support platform 102 at a desired position. A locating fixture 108 is configured to be coupled to the mobile imaging device. The platen and locating fixture are configured to be selectively coupled to one another in a fixed relationship so that the position of the mobile imaging device 104 is fixed with respect to the support platform 102.

The support platform 102 is configured to support a patient. One example of a suitable support platform 102 is a conventional surgical bed. The mobile imaging device 100 in the illustrated embodiment is a C-arm x-ray machine. A C-arm x-ray machine has a C-shaped member 110 that contains an x-ray source 112 and an image receptor 114 mounted on opposing ends of the C-arm 110. X-rays may be emitted by the source 112 so that they are incident upon and detected by the image receptor 114. The source 112 and the image receptor 114 are positioned to irradiate an object, such as a patient, with X-rays and produce image data of the object.

The C-arm member 110 is slidably mounted to a support member 116 so that it is movable in relation to the support member 116. This permits the x-ray source 112 and image receptor 114 to be rotated along the arc of the C-arm. The C-arm support member 110 also permits rotation of the C-arm about its axis 118. The C-arm may be moved up and down along a C-arm column 120. The freedom of movement allows the C-Arm to be configured to obtain desired images.

The imaging system 100 may include encoders and the like to detect the configuration (e.g., the position, rotation and height) of the C-arm, which may be stored in a memory on the imaging system. The imaging system may also store other imaging parameters, such as exposure and collimation. Multiple configurations may be stored for later recall and the configurations may be associated with stored positions. That is, multiple configurations may be stored with a single position of the base so that multiple images may be obtained at a single location without moving the base.

The C-arm column 120 is supported by a base 122 which is mounted on wheels 124, 136 to permit free movement of the base 122. The wheels typically comprise primary wheels 124 and steering wheels 136. The primary wheels are typically fixed into position. The steering wheels 136 may pivot about a vertical axis. This allows a user to move and steer the mobile imaging device 100.

The locating fixture 108 is coupled to the base 122. In one embodiment, the locating fixture 108 comprises pins 126 for engaging apertures 128 in a mating surface of the platen 106. The platen 106 is movably coupled to the surgical bed and may be selectively fixed into position relative to the support platform 102. In one embodiment, the platen 106 engages a guide rail 130 mounted to the side of the support platform 102 so that the platen 106 is slidable along the longitudinal axis of the guide rail 130. The guide rail 130 may comprise a dovetail slot 132 and the platen 106 may have a corresponding dovetail 134 for engaging the slot. A locking device, such as a clamp, may be provided to selectively fix the platen 106 into location with respect to the guide rail 130. One or more additional platens may be coupled to the support platform for selective coupling with the locating fixture. The provision of additional platens allows reproducible images at multiple locations. The guide rail 130 may be provided at either side of the bed, at the foot and head the bed or in any desirable combination thereof.

In operation, the locating fixture 108 is engaged with the platen 106 by inserting the alignment pins into the apertures in the locating fixture. The base 122 of the imaging device 100 is moved into a desired location, and the C-arm 110 is configured to obtain an image. Once the operator determines that the base 122 is properly positioned, the platen 106 may be locked into position. The configuration (e.g., position, rotation, and height) of the C-arm 110 and any desired imaging properties may be stored in memory and associated with the position of the base. Any desired images are then obtained. The imaging device 100 may then be moved away from the support platform 102, thereby disengaging the locating fixture 108 from the platen 106. The imaging device 100 may be stored in any convenient location while one or more medical procedures are performed on the patient. When additional images are desired, the mobile imaging device may be returned to the original location, and the operator engages the locating fixture 108 with the platen 106, thereby placing the base 122 of the imaging device 100 into the same position as when the original images were taken. If the C-arm 110 has been moved with respect to the base 122, the stored data regarding position, rotation and height may be used to reconfigure the C-arm 110 into the same position as the original image. This may be done automatically, using actuators or the like, or manually. After the base is positioned in the same location and the C-arm is configured to the same position, images may be obtained from the patient.

FIG. 3 illustrates another embodiment of a mobile imaging device 300. The mobile imaging device 300 is mounted on a base 302 which may be placed into position adjacent to a support platform 304 to obtain images of a patient on the support platform 304. The base has two primary wheels 306 and two steering wheels 308. In the illustrated embodiment, the primary wheels are coupled to rotational position encoders 310 to detect rotation of the primary wheels 306. The rotational position encoders 310 provide data to a computer 314 with a digital readout 316. The rotational position encoders 310 may be placed on one or more of the primary wheels 306, or may be placed on the steering wheels 308. The primary wheels 306 are typically fixed so that they point in one direction. The steering wheels 308 may be pivoted to provide steering to the mobile imaging device when being moved. The steering wheels 308 are coupled to angular position encoders 312 which provide data to the computer 314.

In operation, the mobile imaging device 300 is placed into a desired location to obtain images. Once in the proper position, the operator interacts with the computer 314 to store the current location. The operator may then move the device away from the support platform to allow medical procedures to be performed on the patient. The computer 314 uses the rotational and angular position encoders 310, 312 to determine the displacement from the stored location. When the operator wishes to move the mobile imaging device 300 back into the original position, the operator may use information displayed visually to move the imaging device. For example, the computer may utilize x and y coordinates corresponding to front to back movement and side to side movement of the mobile imaging device. The x and y coordinates may be set to zero at the original location, and the displacement from the original location may be displayed on the display. Other types of visual indicators or audible indicators may be also used. The computer 314 may store multiple positions which may be recalled independently to allow for taking images at multiple locations.

Video cameras may also be provided on the mobile imaging device or in the operating suite to allow remote monitoring and/or remote operation of the mobile imaging device. In this way, a remote operator could assist a technician, nurse, or other personnel already in the operating suite in properly placing the imaging device, configuring the C-arm and obtaining images.

As illustrated in FIG. 4, a mobile imaging device 400 with angular and rotational position encoders 410, 412 may also include a powered drive capability. In this case, a powered drive unit 420 may supply motive power to either the primary wheels 406 or the steering wheels 408. The operator may use the powered drive to assist in moving the imaging device 400. In addition, a motorized steering mechanism 418 may be provided to adjust the direction of the steering wheels 408. In operation, the computer 414 may control the powered drive and the motorized steering mechanism to move the mobile imaging device to a desired location. For example, the mobile imaging device 400 may be provided with a “home” location, such as a corner of the operating suite, where the mobile imaging device 400 is typically stored. An operator may move the mobile imaging device 400 into a desired location for taking an image, either manually or by using the powered drive unit 420 and motorized steering mechanism 418. The position may be stored into the memory of the computer 414. Once stored, the computer may operate the powered drive unit 420 and motorized steering mechanism 418 to move the mobile imaging device between the home location and stored location. An operator may also store a path for movement between the home location and stored location into the computer 414. Such a path would be useful to avoid having the mobile imaging device inadvertently bump into equipment or people in the operating suite. Safety features, such as contact or proximity sensors, may also be provided to prevent inadvertent contact in this or any other embodiment.

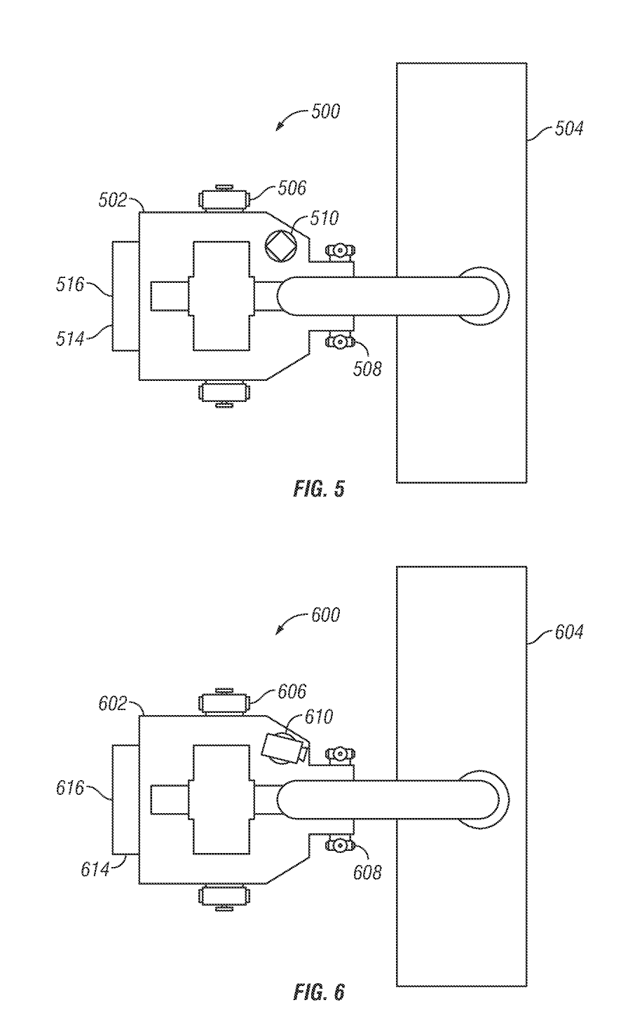

FIG. 5 illustrates another embodiment of a mobile imaging device 500. The mobile imaging device 500 is mounted on a base 502 which may be placed into position adjacent to a support platform 504 to obtain images of a patient on the support platform 504. The base has two primary wheels 506 and two steering wheels 508. An inertial measurement sensor 510 is coupled to the base 502 to provide data to a computer 514 with a digital readout 516. The inertial movement sensor 510 is preferably a MEMS type gyroscope/accelerometer sensor. The data provided by the inertial movement sensor 510 indicates the direction and acceleration of the base and can be used to derive the movement and position of the base 502. Thus, the system may be used as described previously to re-position the mobile imaging device at a desired location. This can be done manually or using a powered drive/motorized steering system as previously described.

FIG. 6 illustrates another embodiment of a mobile imaging device 600. The mobile imaging device 600 is mounted on a base 602 which may be placed into position adjacent to a support platform 604 to obtain images of a patient on the support platform 604. The base has two primary wheels 606 and two steering wheels 608. A video positioning system 610 is coupled to the base 602 to provide data to a computer 614 with a digital readout 616. The video positioning system provides visual data to the computer 614. The system may use the visual data to store an image representative of a particular position. Using this position information, the system may then move and re-position the mobile imaging device. Thus, the system may be used as described previously to re-position the mobile imaging device at a desired location. This can be done manually or using a powered drive/motorized steering system as previously described.

The image data produced by the video positioning system 610 may be transmitted to a remote operator. The operator may also be provided with controls to remotely operate the mobile imaging device, including any powered drive and motorized steering mechanism. This would allow one operator to operate multiple units from one location.

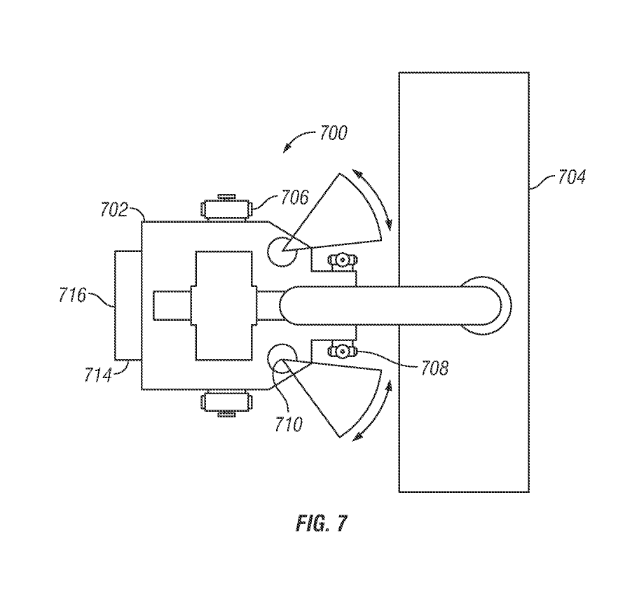

FIG. 7 illustrates another embodiment of a mobile imaging device 700. The mobile imaging device 700 is mounted on a base 702 which may be placed into position adjacent to a support platform 704 to obtain images of a patient on the support platform 704. The base has two primary wheels 706 and two steering wheels 708. A laser displacement instrument 710 is coupled to the base 702 to provide data to a computer 714 with a digital readout 716. The laser displacement system provides data representative of the location of the imaging device 700 to the computer 714. In one embodiment, reflective elements are placed on the support platform 704 to aid in the detection of the placements of the mobile imaging device 700. Using the position information gathered from the laser displacement instrument 710, the system may then move and re-position the mobile imaging device. Thus, the system may be used as described previously to re-position the mobile imaging device at a desired location. This can be done manually or using a powered drive/motorized steering system as previously described.

Combinations of any of the above embodiments are within the scope of the present invention. For example, a system may incorporate inertial movement sensors, encoders, video position system and laser displacement scanners in any combination of one or more. Using a combination of the devices provides more accuracy than using a single device.

The above specification and examples provide a complete description of the structure and use of exemplary embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the present devices are not intended to be limited to the particular forms disclosed. Rather, they include all modifications and alternatives falling within the scope of the claims, and embodiments other than the one shown may include some or all of the features of the depicted embodiment. For example, components may be combined as a unitary structure, and/or connections may be substituted. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. A device for taking images of a patient on a support platform with a mobile imaging device, comprising:
a platen configured to be coupled to the support platform in a fixed position; and
a locating fixture configured to be coupled to the mobile imaging device in a fixed position, wherein the platen and locating fixture are adapted to be selectively coupled to one another in a fixed relationship.

2. The device of claim 1, wherein the support platform comprises a longitudinal axis, and the platen is slidable along the longitudinal axis.

3. The device of claim 1, wherein the support platform comprises a longitudinal slot, and the platen comprises a key for interfacing with the longitudinal slot.

4. The device of claim 3, wherein the longitudinal slot comprises a dovetail slot and the key comprises a dovetail key.

5. The device of claim 1, wherein the platen comprises a mating surface with apertures and the locating fixture comprises pins for engaging the apertures.

6. The device of claim 1, further comprising a locking apparatus for locking the locating fixture into position with respect to the platen.

7. The device of claim 1, further comprising a second platen coupled to the support platform for selective coupling with the locating fixture.

8. An imaging device, comprising:
a mobile base for supporting an imaging apparatus; and
instrumentation for measuring displacement of the mobile base with respect to a patient support platform.

9. The device of claim 8, wherein the instrumentation comprises encoders coupled to wheels coupled to the mobile base.

10. The device of claim 8, wherein the instrumentation comprises a laser displacement instrument.

11. The device of claim 8, wherein the instrumentation comprises an inertial measurement sensor.

12. The device of claim 11, wherein the inertial measurement sensor comprises a gyroscope and an accelerometer.

13. The device of claim 8, wherein the instrumentation comprises a video positioning system.

14. The device of claim 8, further comprising a remote operation controller for operating the imaging apparatus by a remote user.

15. The device of claim 8, further comprising
a memory for storing at least one location of the mobile base;
a powered transport for moving the mobile base; and
a controller for controlling the powered transport to move the mobile base to the at least one stored location.

16. The device of claim 15, wherein the controller may be operated by a remote user.

17. The device of claim 15, wherein the imaging device comprises a C-arm, and the memory is configured to store articulation information regarding the position of the C-arm.

18. The device of claim 17, wherein the memory is configured to store imaging properties of the imaging apparatus.

19. The device of claim 18, wherein the imaging properties comprise an amount of x-ray energy delivered and a collimation of an image.

20. The device of claim 15, wherein the controller is configured to move the mobile base to a storage position.

21. The device of claim 15, wherein the at least one location comprises a plurality of locations, and the controller moves the mobile base to a selected one of the plurality of locations.

22. The device of claim 8, further comprising
a memory for storing at least one location of the mobile base; and
a display for indicating the position of the mobile base with respect to the at least one location.

23. The device of claim 22, wherein the at least one location comprises a plurality of locations, and the display indicates the position of the mobile base with respect to a selected one of the plurality of locations.

24. The device of claim 8, further comprising
a memory for storing at least one location of the mobile base; and
a steering mechanism for guiding the mobile base toward the at least one stored location.

25. The device of claim 24, wherein the at least one location comprises a plurality of locations, and the steering mechanism guides the mobile base toward a selected one of the plurality of locations.

26.-37. (canceled)
